(19) United States
(12) Patent Application Publication
(10) Pub. No.: US 2004/0234560 A1
(43) Pub. Date: Nov. 25, 2004

(54) Cosmetic materials and process for producing the same

(76) Inventors: Akihiko Kimura, Aichi (JP); Yuji Harada, Aichi (JP); Atsushi Takada, Aichi (JP); Asao Shioda, Aichi (JP); Toshitaka Okada, Aichi (JP); Hiroki Yamada, Aichi (JP); Moriji Yamada, Aichi-gun (JP); Itsuko Yamada, Aichi-gun (JP); Kaoru Yamashita, Aichi (JP); Tomohiro Koizumi, Aichi (JP)

Correspondence Address: MORGAN LEWIS & BOCKIUS LLP, 1111 PENNSYLVANIA AVENUE NW, WASHINGTON, DC, 20004, US

(21) Appl. No.: 10/473,719

(22) PCT Filed: Apr. 5, 2002
(86) PCT No.: PCT/JP02/03445

(30) Foreign Application Priority Data
Apr. 6, 2001 (JP) 2001-108337
Dec. 11, 2001 (JP) 2001-377636

(51) Int. Cl. A61K 35/78; A61K 7/00

(52) U.S. Cl. 424/401; 424/757; 435/170; 424/93.46

(57) ABSTRACT

The present invention relates to a naturally derived cosmetic material obtained from bean broth, or by being fermented it or treating it with enzymes, and to a manufacturing method thereof. The method for manufacturing the cosmetic material of the present invention is characterized in that beans are added to a water-based solvent and heated, and the bean broth thus obtained is subjected to fractional purification, to inoculation with Bacillus natto and fermentation culturing, or to the action of soybean Aspergillus bacteria-derived proteases. Because the cosmetic material obtained by this method has excellent SOD activity, cytotonic activity, collagen-formation accelerating ability, antibacterial activity, and tyrosinase inhibiting action, this cosmetic material inhibits the peroxidation of skin lipids and prevents skin aging, and can be appropriately used as a beauty product having excellent functionality in the skin.

### TECHNICAL FIELD

The present invention relates to a cosmetic material and a manufacturing method thereof, and more particularly relates to a naturally-derived cosmetic material obtained by the microfiltration or fractional purification of soybean, black bean, pea, fava bean, adzuki bean, or other bean extract, particularly waste liquid of bean broth, or by fermenting or treating the aforementioned bean extract, particularly waste liquid of bean broth, with enzymes, and to a manufacturing method thereof.

### BACKGROUND ART

Beans are leguminous plants cultivated for the use of their seeds, and have been used in a wide variety of forms since antiquity. Particularly, in addition to being cooked directly for food, beans have broad application to processed food products, and maintain important status as a source of protein and fat. When beans are used as a starting material for processed food products, large quantities of bean broth and steaming broth (these two terms are combined hereinafter and referred to simply as “broth”) are generated during processing, but it is presently common for this broth to be released into the environment as-is.

However, this broth is rich in organic components, and thus spoils easily, and as a result of causing the proliferation of microorganisms along with a foul odor, it causes water contamination when released into the environment in large quantities. At the same time, because large quantities of this broth are generated during processing, purification thereof without placing a burden on the environment requires large amounts of resources and costly equipment.

Known methods of using this waste liquid of bean broth include, for example, a method for manufacturing fermented food products by fermentation whereby Bacillus natto is infused into waste liquid of bean broth (JP-A-56-106565), effective utilization of soybean broth (“Japan Brewers Association” Vol. 92 (7), 1997), and the like, but these methods can be utilized only for food products. Therefore, from the perspective of reducing the discharged amount of waste liquid of bean broth, methods have been sought that would be capable of effectively utilizing this broth not only in the food products industry, but also in an even wider range of applications. On the other hand, as for beans themselves, reports have been made concerning moisture absorption and retention in soy peptides having a molecular weight of 1,000 or less, and their application as cosmetic materials with added functionality (Fragrance Journal, 1994-7), but this method utilizes only the beans themselves, and does not utilize the bean broth, which is a byproduct of food product manufacturing. In addition, no mention is made of a method for utilizing the broth.

Cosmetic products containing placenta extract have also been recently publicized as skin cosmetics (JP-A-2000-16917). Placenta extract is an extract derived from the placenta of cows, pigs, horses, and other livestock, and from human placenta. It possesses not only moisture-retentive effects, but also antioxidant and active oxygen removal effects, and is used as a beauty product for preventing skin aging.

However, while this placenta extract can be appropriately used as a cosmetic material as described above, because it is derived from the placenta of vertebrate animals, it cannot be obtained in large quantities, and thus has the drawback of being naturally expensive. Therefore, development has been sought of a cosmetic material that demonstrates the same effects as placenta extract and is derived from a starting material that can be obtained more easily and in larger quantities than placenta extract.

### SUMMARY OF THE INVENTION

An object of the present invention, developed in view of the circumstances described above, is to provide a naturally-derived cosmetic material obtained by microfiltration or fractional purification of soybean, black bean, pea, fava bean, adzuki bean, and other bean extract, particularly waste liquid of bean broth, or by fermenting or treating the aforementioned bean extract, particularly waste liquid of bean broth, with enzymes, and a manufacturing method thereof.

Superoxide dismutase (hereinafter referred to as “SOD”) is an enzyme that catalyzes the dismutation reaction of superoxide radicals (O₂⁻), generated by the single electron reduction of enzyme molecules, at a near diffusion controlled speed, and that reduces the concentration of O₂⁻ within cells. The type of active enzyme represented by O₂⁻ is usually produced in the body from activated macrophages and other phagocytes, and has bactericidal and tumoricidal effects. However, these types of active enzymes do not have selective toxicity, and are known to induce various disorders in the body as a result of their having effects on normal cells as well. For example, these enzymes have been shown to have disabling effects on cells as a result of membrane damage by peroxidation of lipids, protein conformation change by the oxidation modification of proteins, DNA breakage, and the like. Inhibiting lipid peroxidation, particularly the peroxidation of lipids in the skin, to prevent skin aging have therefore been recently emphasized in the cosmetics industry.

The present inventors previously discovered active enzyme inhibiting effects in a fermented liquid manufactured by inoculating Bacillus subtilis into a medium containing rice bran and soybean, and culturing and filtering the product (JP-A-6-284872), and also discovered blood alcohol concentration reducing effects in a fermented liquid manufactured by inoculating Bacillus subtilis or Bacillus natto into a medium containing rice bran and soybean within a specific pH range (JP-A-3-272657). Upon subsequent investigation of the relationship between beans and their physiological effects, the inventors discovered that extract of soybeans and other beans, and particularly the waste liquid of bean broth that had always been disposed of as waste, have functionality in the skin, and also discovered that this functionality in the skin can be further enhanced by fermenting or enzymatically treating the bean broth with certain bacteria, and thus developed the present invention.

The present invention is as described below.

1. A method for manufacturing a cosmetic material, characterized in adding beans to a water-based solvent and performing extraction, and then microfiltering and/or fraction purifying the bean extract thus obtained.

2. A method for manufacturing a cosmetic material, characterized in adding beans to a water-based solvent and performing extraction, inoculating microorganisms belonging to the genus Bacillus into the bean extract thus obtained, and fermenting and culturing the product.

3. The method for manufacturing a cosmetic material according to claim 2, wherein the microorganisms belonging to the genus Bacillus comprise Bacillus natto or Bacillus subtilis.

4. A method for manufacturing a cosmetic material, characterized in adding beans to a water-based solvent and performing extraction, and then employing enzymes to act on the bean extract thus obtained.

5. The method for manufacturing a cosmetic material according to claim 4, wherein the enzymes comprise proteases derived from soybean Aspergillus.

6. A cosmetic material characterized in being obtained by adding beans to a water-based solvent and performing extraction, and then microfiltering and/or fraction purifying the bean extract thus obtained.

7. A cosmetic material characterized in being obtained by adding beans to a water-based solvent and performing extraction, inoculating microorganisms belonging to the genus Bacillus into the bean extract thus obtained, and fermenting and culturing the product.

8. A cosmetic material characterized in being obtained by adding beans to a water-based solvent and performing extraction, and then employing enzymes to act on the bean extract thus obtained.

By means of the present invention, a cosmetic material derived from plant matter having excellent SOD effects, cytotonic effects, collagen-forming effects, tyrosinase inhibiting action, and the like can be obtained by soybean, black bean, pea, fava bean, adzuki bean, and other bean extract, particularly waste liquid of bean broth, or by being fermented them or treating them with enzymes, proteases, or other enzymes. The cosmetic material obtained by the manufacturing method of the present invention can be appropriately used as a collagen substitute material having moisture absorption and water retention, or as a placenta substitute cosmetic material that demonstrates the same effects as placenta extract. Also, by means of the present invention, because waste liquid of bean broth, which has hitherto been discarded, can be utilized as a starting material, the quantity of discharged waste liquid of bean broth can be reduced, the effects thereof on the environment can be reduced, and the economic burden involved in waste fluid processing can be alleviated.

Furthermore, because byproducts obtained as condensates during the fractionation step in the method for manufacturing the cosmetic material of the present invention are high in nutritional value and do not contain harmful substances, these can be reused in various starting materials for food processing, including health food materials, as well as in animal feed, pet food ingredients, organic fertilizers for gardening, and basic materials for microbial cultures, or as raw materials in cosmetic materials. With the method for manufacturing the cosmetic material of the present invention as described above, there are almost no substances disposed of as industrial waste, so the levels of BOD and COD in the final wastewater can be kept at the industrial wastewater standard of 10 ppm or less, which can contribute to both the effective utilization of unused resources and to environmental preservation.

### DISCLOSURE OF THE INVENTION

The method for manufacturing a cosmetic material according to claim 1 is characterized in adding beans to a water-based solvent and performing extraction, and then microfiltering and/or fraction purifying the bean extract thus obtained. The cosmetic material obtained by this method can be produced with ease and in large quantities from beans (plant material) as the starting material thereof, and can function as the raw material for the cosmetic material of the present invention. Also, because it has excellent SOD activity, this material can function as a cosmetic material for inhibiting the peroxidation of lipids in the skin and preventing skin aging. Also, the cosmetic material obtained by this method has cytotonic activity and functions to accelerate collagen formation, and because of the excellent antibacterial properties thereof, the material can be appropriately used as a collagen substitute material, or as a placenta substitute cosmetic material that demonstrates the same effects as placenta extract. Furthermore, the cosmetic material obtained by this method can be appropriately used as a whitening cosmetic material, due to the excellent tyrosinase inhibiting action thereof.

In the present invention, the beans are not particularly limited in terms of type, insofar as the beans consist of the seeds of leguminous plants generally used for food and animal feed, and include, for example, soybeans, black beans, peanuts, adzuki beans, black-eyed peas, kidney beans, fava beans, peas, green beans, coffee beans, cocoa beans, sesame seeds, sunflower seeds, and the like. Defatted beans, defatted seeds, kina powder, bean powder, bean chaff, and hydrosylates thereof can also be used. Water (including hot water) is also usually used as the water-based solvent, but insofar as the quality of the bean extract is maintained, it is also possible to use ethyl acetate, alcohol (ethanol and the like), and other hydrophilic organic solvents, as well as solvent mixtures and other preparations of these organic solvents, particularly hydrophilic organic solvents, with water or hot water. It is further possible to use waste liquid of bean broth generated in the usual process involved in bean processing, as well as leachates generated as byproducts of tofu curdling processes and the like.

In the cosmetic material according to claim 1, beans are added to the aforementioned water-based solvent, and bean extract is obtained by performing extraction. In this case, extraction may be performed at ambient temperature or with heating. In particular, this arrangement is preferred when the bean extract referred to herein is a heat-extracted bean broth, particularly waste liquid of bean broth generated in the usual process involved in bean processing, or a leachate generated as a byproduct of tofu curdling processes or the like, because in this case the waste liquid of bean broth and leachates that were traditionally disposed of can be utilized effectively. The method and conditions for obtaining such bean extract is not particularly limited. For example, the beans may be used as-is, and can be crushed and used as needed, and insofar as the quality of the bean extract can be maintained, removal of impurities or other preprocessing may be performed. Furthermore, in the case of heat extraction, the heating temperature and heating time used for obtaining the bean extract may consist of a variety of conditions in a range that allows bean components to be adequately extracted and the desired quality thereof to be maintained. For example, the heating temperature may be such that the heat added usually brings the temperature of the water-based solvent to 40 to 100° C., preferably 50 to 80° C., and more preferably 50 to 70° C. Also, the pH of the water-based solvent is usually 3 to 7, preferably 4 to 6, and more preferably 4 to 5. Setting the heating temperature and pH to within these ranges is preferred, because extraction can thus be performed efficiently. Also, the bean extract can be used as-is, or insofar as the quality thereof is maintained and no cost in waste processing is required, legally permitted additives may also be used for the purpose of increasing the efficiency of extraction and waste processing of the target substance.

In the cosmetic material according to claim 1, after beans are added to the aforementioned water-based solvent and bean extract is obtained by extraction, the bean extract is subjected to microfiltration or fractional purification. For example, microfiltration may be performed using a filtration membrane that is usually 0.1 to 1.0 μm, preferably 0.3 to 0.6 μm, and more preferably 0.3 to 0.45 μm, after foreign matter, impurities, and other solids have been removed from the aforementioned bean extract by filter cloth filtration, press filtration, centrifugation, or the like. In this case, the material constituting the filtration membrane is not particularly limited. Vacuum concentration, membrane concentration, or another concentration procedure may be performed following microfiltration, and a material for use in a beauty product can be obtained by means of drying the product by freeze-drying, heat drying, or the like as needed. Also, a gel filtration method, ultrafiltration method, or other means may be employed as needed for fractional purification. In this case, it is preferable to use a molecular sieve with a molecular weight (hereinafter referred to as “MW”) range of 1,000 to 10,000, preferably 3,000 to 10,000, more preferably 3,000 to 8,000, even more preferably 3,000 to 7,000, particularly preferably 4,000 to 7,000, and most preferably 5,000 to 6,000. It is preferable to perform fractional purification by a molecular sieve of this range, because bean allergies can thus be minimized. Also, the remaining concentrate can be utilized as a raw cosmetic material, and can be used for applications other than cosmetics (for example, food materials, beverage or food additive materials, textile processing materials, or the like).

Also, one or the other of the aforementioned microfiltration and fractional purification may be performed alone, or both may be performed. By this means, activity can also be further enhanced.

The method for manufacturing the cosmetic material according to claim 2 is characterized in adding beans to a water-based solvent and performing extraction, inoculating microorganisms belonging to the genus Bacillus into the bean extract thus obtained, and fermenting and culturing the product. The cosmetic material obtained by this method can function as a cosmetic material with appeal particularly in prevention of skin aging, prevention of skin roughness, and the like, and is thus preferable due to the strong SOD functional activity, cytotonic effects, and collagen-forming effects thereof. Also, the cosmetic material obtained by this method can be appropriately used as a whitening cosmetic material, due to the excellent tyrosinase inhibiting action thereof.

The “microorganisms belonging to the genus Bacillus” used in the method for manufacturing the cosmetic material according to claim 2 may consist of microorganisms belonging to the bacteriological genus Bacillus, and may include, for example, Bacillus natto or Bacillus subtilis, as described in claim 3. This arrangement is preferable because proteases and various other useful enzymes are produced outside the microorganisms in fermentation by Bacillus natto or Bacillus subtilis, and these enzymes can be recovered simultaneously during fractionation. Usually, commercially available regular Bacillus natto and Bacillus subtilis are used, but other than these, mutant strains of Bacillus natto and Bacillus subtilis with altered bacteriological properties obtained naturally or by means of nitrosoguanidine or other chemical substances, X-rays, ultraviolet rays, or other artificial mutating means may also be utilized, insofar as the properties that give rise to the functional material possessed by the cosmetic material of the present invention (described in detail hereinafter) having SOD effects are not compromised. Also, because the culture solution used consists of waste liquid of soybean broth, and is usually discharged under normal conditions, related bacteria having more robust fertility in a natural environment are preferred for the Bacillus natto or Bacillus subtilis.

In the method for manufacturing the cosmetic material according to claim 2, the method for obtaining the bean extract can be the same as the aforementioned method for manufacturing the cosmetic material according to claim 1. In this case, before the “microorganisms belonging to the genus Bacillus” are inoculated into the aforementioned bean extract and fermentation culturing is performed, bactericidal processing can be performed for the bean extract as needed for killing off contaminant bacteria in the bean extract intermixed in the starting material itself, or in the bean extract preparation. This bactericidal processing can be performed by treatment involving antibacterial filtering, and by heat-treating (steaming, for example) the bean extract. The heat treatment is usually performed at 100 to 130° C. for 1 to 60 minutes, but because Bacillus natto has generally robust fertility for a bacterium of the genus Bacillus, complete sterilization at high temperature and high pressure (121° C./15 minutes) is not required when the medium is heat-sterilized, and the usual steam/pressure heat sterilization by increasing the quantity of inoculated seed bacteria may be used. On the other hand, bean broth that is heat-treated directly after discharge may also be used without additional heat sterilization after prompt cooling to a temperature at which Bacillus natto and the like can grow easily, due to the relative scarcity of contaminant bacteria therein.

In the method for manufacturing the cosmetic material according to claim 2, after the aforementioned sterilization treatment is performed as needed, the “microorganisms belonging to the genus Bacillus” are inoculated into the bean extract, and fermentation culturing is performed. Usually in this case, the “microorganisms belonging to the genus Bacillus” are inoculated directly into the bean extract and cultured, although it is also possible to inoculate the bean extract and the “microorganisms belonging to the genus Bacillus” into a medium in which the “microorganisms belonging to the genus Bacillus” can grow, and to perform culturing. The medium in this case may consist of any medium in which the “microorganisms belonging to the genus Bacillus” can grow, and may be either a liquid or solid medium. Also, as described above, due to the relative scarcity of contaminant bacteria in a bean broth that is heat-treated directly after discharge, pure cultured “microorganisms belonging to the genus Bacillus” (Bacillus natto and other related bacteria) may be inoculated therein and cultured without additional heat sterilization. Specifically, an inoculation method similar to the Bacillus natto inoculation process involved in natto manufacturing may also be employed.

Also, in the method for manufacturing the cosmetic material according to claim 2, a method of directly adding bacteria or a bacteria-containing solution to the bean extract or to the aforementioned medium is usually cited as a method for inoculating the “microorganisms belonging to the genus Bacillus,” but a method may also be used in which the “microorganisms belonging to the genus Bacillus” are immobilized on an appropriate carrier. Such a method is preferable because microorganisms can be reused after fermentation is complete. Universal immobilization methods for immobilizing microorganisms in a polymer matrix, carrier binding methods for chemically binding microorganisms directly to an immobilized carrier, cross-linking methods for insolubilizing by cross-linking microorganisms to each other, and other conventional methods are included as this microbial immobilization method. Among these, universal immobilization methods that have little effect on the microorganisms are suitable. These universal immobilization methods include a lattice-type method in which microorganisms are enclosed in a fine lattice in a polymer gel, and a microcapsule-type method for coating the microorganisms. Among these methods, a lattice-type universal immobilization method is preferable from the perspective of maintaining microbial activation and because of the ease of immobilization.

Also, the fermentation culturing methods and conditions are not particularly limited insofar as fermentation is performed, and various conditions can be set according to the growth characteristics of the bacteria used. Usually, fermentation culturing is performed by ventilation and stirring, and the culturing temperature is around 40 to 45° C. Also, the pH of the medium is not particularly limited, but is usually 4 to 7, preferably 5 to 7, and more preferably 6 to 7. When the pH of the medium is adjusted, sodium bicarbonate or the like may be used as an alkaline agent. Proteases may also be added to the medium starting material. This is useful in this case, because peptides in the beans are further dissociated. Also, during culturing, various nutrient sources (nitrogen sources, carbon sources, minerals, vitamins, and the like) and the like can also be added to the bean extract or to the aforementioned medium to increase culturing efficiency and recovered quantity of the target substance. One, two, or more types of glucose, dextrin, lactose, starch, or the like, for example, can be used as carbon sources. Furthermore, the culturing time is also not limited by the fertility of bacteria themselves, but is usually 24 to 72 hours, and preferably 48 to 72 hours.

In the method for manufacturing the cosmetic material according to claim 2, centrifugation, filter pressing, or the like can be performed following fermentation culturing in accordance with the viscosity and other physical properties of the fermentation culture solution in order to remove solids and bacteria. Also, press filtration, microfiltration (0.45 μm), freeze-drying, or other various processes may be performed for the fermentation culture solution. Furthermore, because enzymes having protease activity, as well as various useful extrabacterial enzymes, are produced in the polymer fraction of the fermentation culture solution, these useful extrabacterial enzymes can be recovered by fractionation. Particularly, an arrangement in which fractionation is performed by MW5000 ultrafiltration is preferred because bacteriostatic action and SOD activity are increased, hypoallergenicity can be ensured, and fractional recovery of a cosmetic material fraction having an enzymatically active concentrated fraction and antibacterial activity can be performed.

The method for manufacturing a cosmetic material according to claim 4 is characterized in adding beans to a water-based solvent and performing extraction, and then employing enzymes to act on the bean extract thus obtained. In the method for manufacturing a cosmetic material according to claim 4, the method for obtaining the bean extract can be the same method as the aforementioned method for manufacturing the cosmetic material according to claim 1. Also, in the same manner as in the case of the method for manufacturing the cosmetic material according to claim 2, the bean extract can be subjected to bactericidal processing as needed for killing off contaminant bacteria in the bean extract intermixed in the starting material itself, or in the bean extract preparation, before enzymes are employed in the bean extract. Furthermore, in the method for manufacturing the cosmetic material according to claim 4, after the enzymes have acted, microfiltration or ultrafiltration steps can be performed in the same manner as in claims 1 and 2.

In the method for manufacturing the cosmetic material according to claim 4, the enzymes can be selected as needed from fractions containing low-molecular-weight components that may be useful for the cosmetic materials in the bean extract. For example, in the treatment with amylase, cellulase, pectinase, or the like, isoflavones resembling female hormones (placenta-like hormones), saponins with antioxidant effects, and other fractions may be obtained by hydrolysis of complex polysaccharides. Also, because little formation of bitter peptides occurs when proteases are used, these can be appropriately used for lipstick and other cosmetic materials applied to the mouth area. Herein, the type and origin of the proteases is not particularly limited, insofar as the proteases consist of enzymes having catalyzing effects on the peptide bond hydrolysis reaction. For example, proteases derived from microorganisms belonging to the genus Bacillus, proteases derived from soybean Aspergillus, and the like can be used. Among these, proteases derived from soybean Aspergillus have strong hydrolyzing effects into amino acids, and possess activity in the neutral to weakly acidic pH range (pH 6.0), allowing a cosmetic material to be extracted from broth in its natural state. Thus, when proteases derived from soybean Aspergillus are used, a cosmetic material having UV blocking effects and anti skin allergy effects can be obtained as a cosmetic material used in specialized applications for people whose skin is hypersensitive to ultraviolet radiation, and for people with hypersensitive allergic reactions to clothing and other skin stimulation. Also, the enzymes may be used singly or in combinations of two or more types thereof.

In the method for manufacturing the cosmetic material according to claim 4, when proteases and other aforementioned enzymes are made to act on the bean extract, this action is usually achieved by adding proteases and other of the aforementioned enzymes, or a solution containing proteases and other of the aforementioned enzymes, to the bean broth, but an appropriate carrier with the aforementioned enzymes immobilized thereon may also be used. Using this method is preferred, because microorganisms can be reused after fermentation is completed. Methods for immobilizing the proteases and other aforementioned enzymes include the aforementioned universal immobilization methods, carrier binding methods, cross-linking methods, and other conventionally practiced, publicly known methods. Also, after making proteases and other aforementioned enzymes to act on the bean extract, the product is usually heated, and enzyme activity is stopped.

In the method for manufacturing the cosmetic material of the present invention, after fractional purification, fermentation, or enzyme treatment, the product can be made into a final product as-is, or can be subjected to freeze-drying or another publicly known drying process as needed. Also, the pH may be adjusted as needed in the final product step. Furthermore, in the same manner, in the method for manufacturing the cosmetic material of the present invention, decolorizing by active carbon or the like, filtering by a filtration auxiliary, centrifugation, vacuum concentration, concentration by molecular sieve, or the like can also be performed if necessary during intermediate processes. Also, as a means for maintaining the stability and safety of the final product, heat sterilization, ultraviolet sterilization, and legally permitted bactericides and other additives may be used.

The cosmetic material of the present invention is characterized in being obtained by the method for manufacturing the cosmetic material of the present invention. Because the cosmetic material of the present invention consists of a vegetable starting material and is obtained easily and in large quantities with beans as a starting material, this cosmetic material can be used as a placenta substitute that is capable of bestowing hypoallergenicity and possesses moisture absorption and water retention properties, or as a placenta-substitute cosmetic material that demonstrates the same effects as placenta extract. Also, because the cosmetic material of the present invention has excellent SOD activity, this cosmetic material can be appropriately used as a beauty product for inhibiting peroxidation of skin lipids and for preventing skin aging. Furthermore, because the cosmetic material of the present invention has excellent tyrosinase inhibiting action, this cosmetic material can be appropriately used as a whitening cosmetic material. Also, the cosmetic material of the present invention can be made to contain polypeptides with relatively high molecular weight.

Specific examples of the cosmetic material of the present invention are not particularly limited, and may consist of a water-based solution or broth solution; a powder obtained by drying a liquid cosmetic material by various methods or the like and crushing the product, or impregnating a liquid-absorbent powder; a granulated pellet; a tablet with fillers and other powder components admixed therein; a microcapsule, or the like. Also, other substances may be added to the functional material of the beauty product of the present invention, insofar as the characteristics thereof are maintained. For example, cornstarch or another component with abundant water solubility can be added to make quantitative measurement easier during manufacturing. Specifically, not only products that are wholly composed of the cosmetic material, but also products that contain the cosmetic material as a component are included in the cosmetic material of the present invention.

Another feature of the present invention is that various modified examples can be included in the scope of the present invention according to the object and application thereof without being limited by the specific examples described below. For example, insofar as no additional burden is imposed on the quality of the bean broth and waste processing, organic solvents that are allowed by food hygiene law, pH adjusting agents, and other food additives may be used.

### BRIEF DESCRIPTION OF THE DRAWINGS

FIG. 1 is a preparation process chart for the cosmetic material in Example 1.

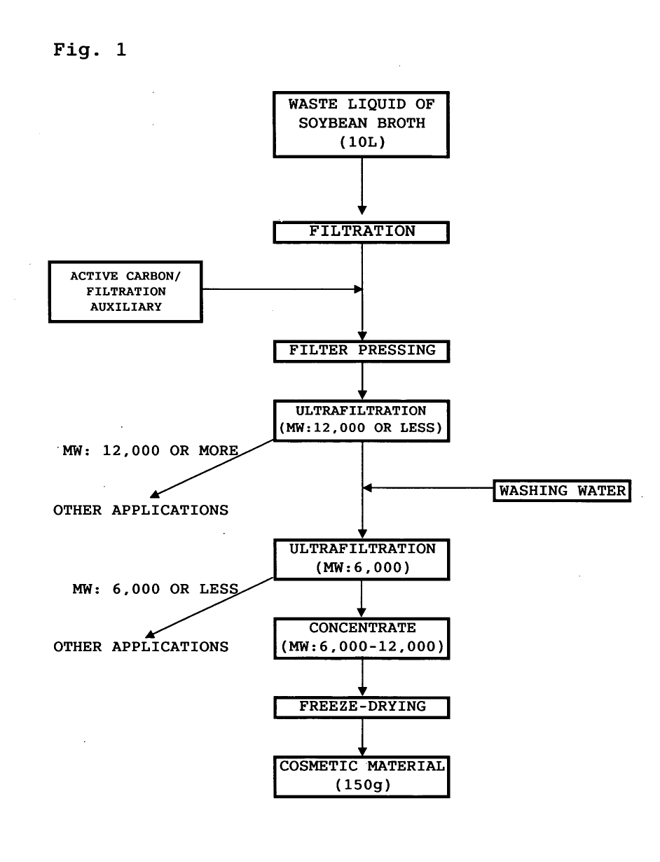

FIG. 2 is a preparation process chart for the cosmetic material in Example 2.

FIG. 3 is a preparation process chart for the cosmetic material in Example 5.

FIG. 4 is a preparation process chart for the cosmetic material in Example 6.

FIG. 5 is a preparation process chart for the cosmetic material in Example 7.

### BEST MODE FOR CARRYING OUT THE INVENTION

(1) Cosmetic Material Preparation 1

### EXAMPLE 1

Waste liquid of soybean broth (wastewater after boiling 100% soybeans in hot water at 100° C. for 30 minutes) obtained during usual manufacturing of soybean/boiled bean processed food products was used as a starting material. 10 liters of the waste liquid of soybean broth were first sieved to remove impurities, and the product was decolorized and filtered by means of a filter press using active carbon and a filtration auxiliary (“Parlite No. 4180,” manufactured by Daika Lion Oriental, Inc.). The transparent filtrate thus obtained was subsequently ultrafiltrated in two steps with a hollow fiber membrane, and the cosmetic material of Example 1 (150 g) was obtained by freeze-drying the product.

### EXAMPLE 2

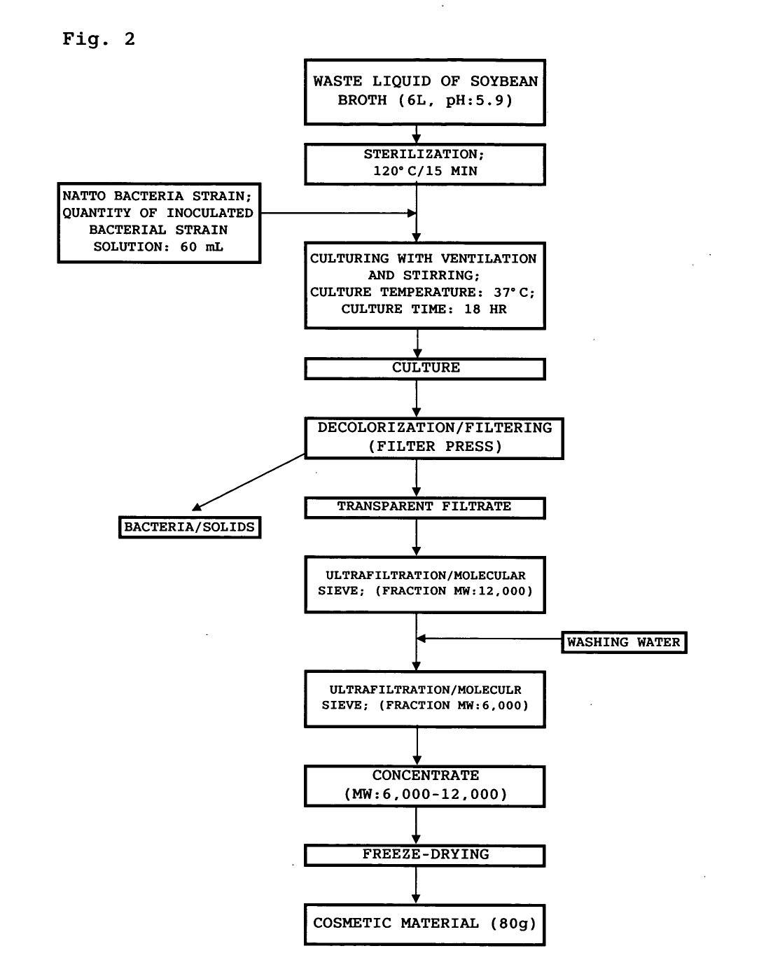

The waste liquid of soybean broth of the aforementioned Example 1 was used as a starting material. Sterilization at 120° C. for 15 minutes was then performed. The sterilized waste liquid of soybean broth was allowed to cool to room temperature, inoculated with commercially available Bacillus natto (manufactured by Takahashi Research Institute), and cultured with ventilation and stirring for 18 hours at 37° C. Also, the pH during culturing was unadjusted. The culture was then heat-sterilized at 90° C. for 30 minutes, filter-pressed using the active carbon and filtration auxiliary of the aforementioned Example 1, and subjected to decolorization and sterile filtration. Ultrafiltration was then performed using a hollow fiber membrane, and the cosmetic material of Example 2 (80 g) was obtained by freeze-drying the product. The process described above is shown in FIG. 2.

### EXAMPLES 3 AND 4

The waste liquid of soybean broth of the aforementioned Example 1 was used as a starting material. 3.5 liters of the waste liquid of soybean broth were first sieved and impurities were removed. Neutral protease (“Protease A,” manufactured by Amano Pharmaceutical, Inc.) and alkaline protease (“Orientase 22BF,” manufactured by Hankyu Kyoei Bussan, Inc.) produced by Aspergillus orizae were then added as soybean Aspergillus-derived enzymes to the aforementioned waste liquid of soybean broth and reacted at 60° C. for five hours, and the system was subsequently heated to 90° C. for five minutes to perform enzyme deactivation treatment. Filter pressing was then performed by the same method as in Example 2, ultrafiltration was carried out using a hollow fiber membrane, and the cosmetic materials (80 g) of Example 3 (neutral protease) and Example 4 (alkaline protease) were obtained by freeze-drying the product.

Also, the waste liquid of soybean broth used as a starting material in the aforementioned Examples 1 through 3 was used in Comparative Example 1. Soybean protein treated with alkaline protease was also used in Comparative Example 2.

(2) Cosmetic Material Functionality Test

Examples 1 through 4 and Comparative Examples 1 and 2 were tested for functionality by the method shown below.

[SOD Activity]

SOD activity was measured by the NBT reduction method using an “SOD Test Wako” manufactured by Wako Pure Chemical Industries, Inc. (in vitro diagnostic agent, authorization code: (63AM) No. 0285). Results thereof are shown as inhibition rates (%). These measurement results are shown hereinafter in Table 1.

(3) Cosmetic Material Preparation 2

### EXAMPLE 5

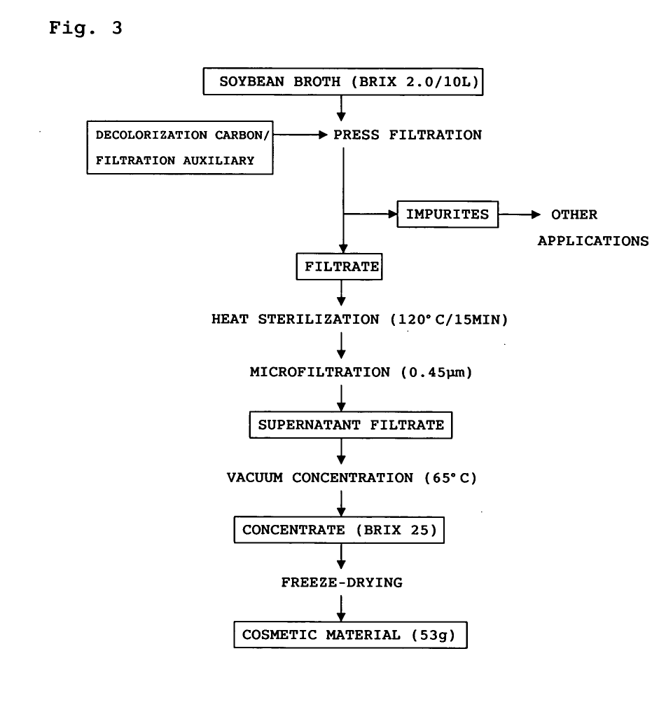

Waste liquid of soybean broth (wastewater after boiling 100% soybeans in hot water at 100° C. for 30 minutes) obtained during usual manufacturing of soybean/boiled bean processed food products was used as a starting material. 10 liters of the waste liquid of soybean broth were first sieved to remove impurities, and the product was decolorized and filtered by means of a filter press using active carbon and a filtration auxiliary (active carbon and diatomaceous earth, manufactured by Wako Pure Chemical Industries, Inc.). The transparent filtrate thus obtained was heat-sterilized (120° C./15 minutes) and microfiltered (membrane thickness: 0.45 μm). After vacuum concentrating (65° C.) the filtrate to a sugar concentration (brix) of about 25, the cosmetic material (53 g) of Example 5 was obtained by freeze-drying the product. The process described above is shown in FIG. 3.

### EXAMPLE 6

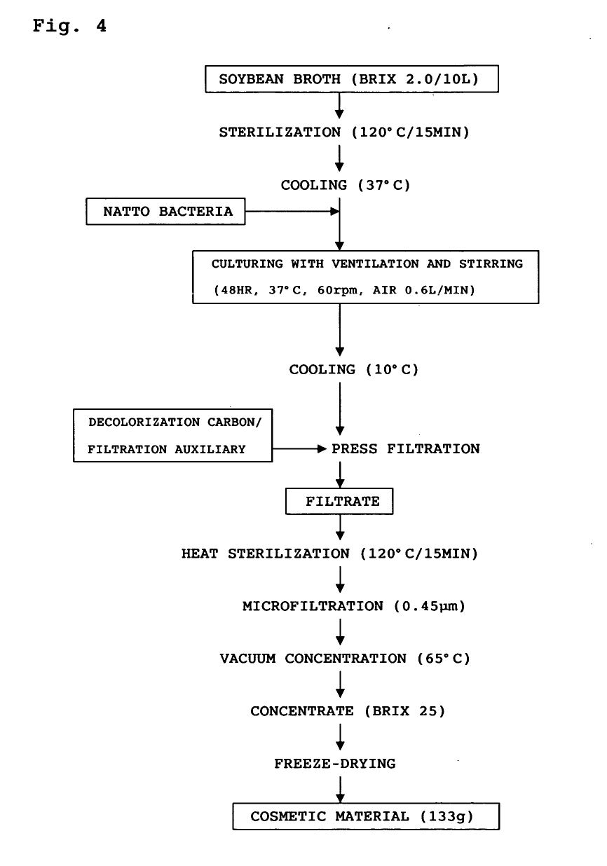

The same waste liquid of soybean broth as in the aforementioned Example 5 was used as a starting material. 10 liters of the waste liquid of soybean broth were first sieved to remove impurities, and sterilization at 120° C. for 15 minutes was then performed. The sterilized waste liquid of soybean broth was allowed to cool to room temperature, inoculated with commercially available Bacillus natto (manufactured by Takahashi Research Institute), and cultured with ventilation and stirring for 48 hours at 37° C. The culture was then cooled to approximately 10° C., subjected to decolorization and filtration by filter pressing in accordance with the same method as in Example 5, and subjected to decolorization and sterile filtration. The filtrate was heat-sterilized, microfiltered (membrane thickness: 0.45 pm), vacuum concentrated, and freeze-dried, yielding the cosmetic material (133 g) of Example 6. The process described above is shown in FIG. 4.

### EXAMPLE 7

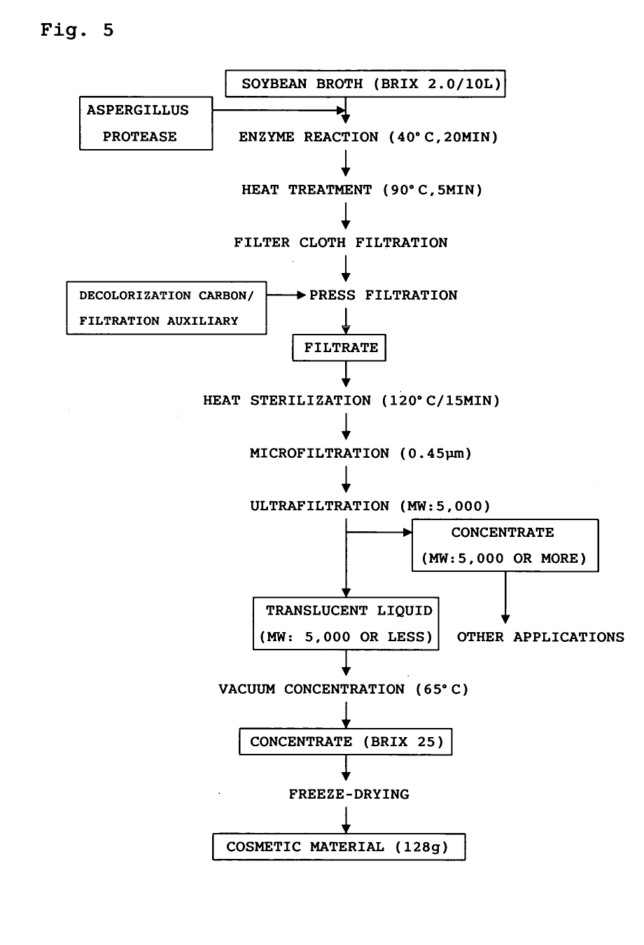

The waste liquid of soybean broth of the aforementioned Example 5 was used as a starting material. Soybean Aspergillus-derived neutral protease (“Protease A,” manufactured by Amano Pharmaceutical, Inc.) was then added to 10 liters of the waste liquid of soybean broth, the product was reacted at 40° C. for 20 minutes, the system was then kept at 90° C. for 5 minutes, and an enzyme deactivation treatment was performed. Decolorization filtration and microfiltration were then performed by filter pressing in accordance with the same method as in Examples 5 and 6, the product was fractionated by MW5000 ultrafiltration, the translucent fluid was vacuum concentrated, the product was freeze-dried, and the cosmetic material of Example 7 (128 g) was obtained. The process described above is shown in FIG. 5.

Also, commercially available hydrolyzed soybean peptide (MW: approx. 1,000) was used in Comparative Example 3; commercially available collagen for cosmetic applications (MW: 4,000 to 5,000) was used in Comparative Example 4; and commercially available silk peptide for cosmetic applications (MW: 500 to 1,500) was used in Comparative Example 5.

(4) Cosmetic Material Functionality Test 2

Examples 5 through 7 and Comparative Examples 3 through 5 were tested for functionality by the method shown below.

[SOD Activity]

Measurement was performed by the same method as in the aforementioned Examples 1 through 4 and Comparative Examples 1 and 2. Results thereof are shown as inhibition rates (%).

[Method for Testing Cytotonic Effects and Collagen-Forming Effects]

1,000 human skin fibroblasts were inoculated onto a 96-well plate and cultured for five days in a 5% FBS-added MEM medium, the medium was replaced with a serum-free MEM medium, and the product was cultured for another day. The cosmetic material of Examples 5 through 7 was then added to a 0.5% serum-added MEM medium to obtain the concentrations shown in Table 2, the product was replaced with a prepared sample-added solution, and culturing was continued for another five days. Based on the Tongrentang cell counting kit 8, viable cell count was then determined by measuring at 450 nm the water-soluble formazan reduced and formed by the dehydrogenase inside the viable cells, with WST-8 tetrazolium salt as a coloring substrate, and the activation effects were then evaluated by finding each corresponding value. One lot of the same product as in the aforementioned test of cytotonic effects was furthermore prepared, and the amount of collagen was found at the end of culturing by using a collagen stain kit (manufactured by Collagen Technical Research) according to the manual included with the kit.

[Test of Antibacterial Activity]

Antibacterial activity testing was conducted based on JIS regulations (L-1902) for the cosmetic material of the aforementioned Example 6. Specifically, antibacterial strength was evaluated by using Staphylococcus aureus at an inoculated bacteria concentration of 5.7×10⁵ units/mL as a test bacterial strain and by measuring the viable bacterial count after soaking a JIS regulation white wool cloth with a specimen containing 10% of the cosmetic material of the aforementioned Example 6. An attached white cloth not soaked with the cosmetic material of the aforementioned Example 6 was used as a control product (Comparative Example 6). Also, the reason that wool was used for the attached white cloth was that bacteria deposit more readily in wool and other natural fibers than in polyester and other synthetic fibers. Results thereof are shown in Table 3 below.

[Tyrosinase Inhibition Test]

In the present test, the tyrosinase inhibition rate (%) was measured by using the cosmetic materials of Examples 5 and 6 and Comparative Examples 3 through 5, with tyrosine as the substrate, and by measuring light absorbance at a wavelength of 475 nm in dopachrome, which is an intermediate in the melanin formation pathway. Specifically, in the present test, tyrosinase (manufactured by Sigma Chemical Co.) obtained from mushrooms was used to prepare a tyrosinase solution (1,200 U/mL) by dissolving the tyrosinase in a McIlvaine buffer solution (a pH 6.8 solution prepared from 0.1 M citric acid solution and 0.2 M sodium hydrogen phosphate solution). Also, a tyrosine solution (tyrosine concentration: 1.66 mM) was prepared by dissolving tyrosine in 1,300 μL of the aforementioned McIlvaine buffer solution.

585 μL of a 1% sample solution of the cosmetic materials of Examples 5 and 6 and Comparative Examples 3 through 5 dissolved in 1,300 μL of the aforementioned tyrosine solution were then added. 130 μL of the aforementioned tyrosinase solution were then added, and incubation was performed at 37° C. for 10 minutes. A reaction solution was then prepared as a measurement specimen by adding 65 μL of a 1.0 M sodium azide stopping solution. Also, a control was prepared by performing operations according to the same procedure as described above, except that 585 μL of water were added instead of 1% sample solution. A blank for the aforementioned reaction solution and control was also prepared in accordance with the aforementioned procedure by adding the aforementioned stopping solution before the aforementioned tyrosinase solution. Light absorbance at 475 nm was also measured for the aforementioned reaction solution and control solution, and for the aforementioned reaction solution and control solution blank. Based on this light absorbance, the tyrosinase inhibition rate (%) was found according to the equation below for the cosmetic materials of Examples 5 and 6 and Comparative Examples 3 through 5. Results thereof are shown in Table 4.

A=[(d−a)−(b−c)]×100/(d−a)

A: tyrosinase inhibition rate (%)

a: light absorbance of control blank

b: light absorbance of aforementioned reaction solution

c: light absorbance of aforementioned reaction solution blank

d: light absorbance of aforementioned control

TABLE 1

| | SOD ACTIVITY (%) |
|---|---|
| EXAMPLE 1 | 42.8 |
| EXAMPLE 2 | 55.7 |
| EXAMPLE 3 | 51.1 |
| EXAMPLE 4 | 44.1 |
| COMPARATIVE | 30.4 |
| EXAMPLE 1 | |
| COMPARATIVE | 41.4 |
| EXAMPLE 2 | |

TABLE 2

| | | VIABLE | COLLAGEN | |
|---|---|---|---|---|
| | | CELL COUNT | QUANTITY | |
| | | (CORRE- | (CORRE- | SOD |
| | | SPONDING | SPONDING | ACTIVITY |
| | | VALUE) | VALUE) | (%) |
| EXAMPLE 5 | 0.01% | 1.39 | 1.50 | 38.4 |
| | 0.05% | 1.48 | 1.29 | |
| EXAMPLE 6 | 0.01% | 1.72 | 1.63 | 45.8 |
| | 0.05% | 1.97 | 1.24 | |
| EXAMPLE 7 | 0.01% | 1.39 | 1.24 | 34.0 |
| | 0.05% | 1.40 | 1.16 | |
| COMPARATIVE | 0.01% | 1.08 | 1.45 | 8.5 |
| EXAMPLE 3 | 0.05% | 1.20 | 1.10 | |
| COMPARATIVE | 0.01% | 1.26 | 1.60 | 16.9 |
| EXAMPLE 4 | 0.05% | 1.42 | 1.23 | |
| COMPARATIVE | 0.01% | 1.23 | 1.62 | 30.5 |
| EXAMPLE 5 | 0.05% | 1.66 | 1.35 | |

TABLE 3

| | | | VIABLE | BACTERIO- |
|---|---|---|---|---|
| | | TIMES | BACTERIAL | STATIC |
| TEST SAMPLE | | WASHED | COUNT (×10⁵) | ACTIVITY |
| A | MEAN VALUE FOR | — | 1 | — |
| | NUMBER OF | | | |
| | BACTERIA | | | |
| | RECOVERED AFTER | | | |
| | INOCULATION OF | | | |
| | CONTROL CLOTH | | | |
| B | MEAN VALUE FOR | — | 75 | — |
| | NUMBER OF | | | |
| | BACTERIA | | | |
| | RECOVERED AFTER | | | |
| | 18 HOURS OF | | | |
| | CULTURING ON | | | |
| | CONTROL CLOTH | | | |
| C | COMPARATIVE | 0 | 520 | −0.9 |
| | EXAMPLE 6 | | | |
| | EXAMPLE 6 | 0 | 120 | 0.8 |

TABLE 4

| | | | COM- | COM- | COM- |
|---|---|---|---|---|---|
| | | | PARA- | PARA- | PARA- |
| | | | TIVE | TIVE | TIVE |
| | EXAMPLE | EXAMPLE | EXAM- | EXAM- | EXAM |
| | 5 | 6 | PLE 3 | PLE 4 | PLE 5 |
| TYROSINASE | 65 | 72 | 0 | 0 | 0 |
| INHIBITION | | | | | |
| RATE (%) | | | | | |

### (5) EFFECTS OF THE EXAMPLES

As shown in Table 1, the SOD activity was 42.8% in Example 1, in which the soybean broth was subjected to fractional purification. SOD activity was also 44.1% in Example 4, in which the soybean broth was treated with alkaline protease, and 51.1% in Example 3, in which neutral protease treatment was performed, indicating that higher SOD activity was obtained herein than in Example 1. Furthermore, in Example 2, in which the soybean broth was cultured with Bacillus natto, SOD activity was markedly higher than in Example 1, at 55.7%, and excellent SOD effects could be seen. In contrast, SOD activity was a low 30.4% in Comparative Example 1, in which the soybean broth was not treated. Also, although SOD activity was somewhat elevated at 41.4% in Comparative Example 2, in which soybean protein obtained from beans themselves was treated with alkaline protease, the SOD activity still remained low. Based on these results, it was apparent that the cosmetic material of the present invention has excellent SOD activity, minimizes formation of peroxidized lipids, prevents skin aging, and possesses characteristics capable of maintaining supple skin.

Also, as shown in Table 2, the SOD activity is high at 34.0 to 45.8% in Example 5, in which the soybean broth was subjected to fractional purification; in Example 6, which was obtained by culturing with Bacillus natto; and in Example 7, in which a neutral protease treatment was performed. Particularly in Example 6, the SOD activity is an extremely high 45.8%, and excellent SOD effects can be seen. In contrast, it is apparent that SOD activity is low, at 8.5 to 30.5%, in Comparative Example 3, in which commercially available hydrolyzed soybean peptides are used; in Comparative Example 4, in which commercially available collagen for cosmetic applications is used; and in Comparative Example 5, in which commercially available silk peptides for cosmetic applications are used.

Furthermore, as shown in Table 2, a comparison of collagen production shows 1.24 to 1.63 at a concentration of 0.01% and 1.16 to 1.29 at a concentration of 0.05% in Examples 5 through 7; and 1.45 to 1.62 at a concentration of 0.01% and 1.10 to 1.35 at a concentration of 0.05% in Comparative Examples 3 through 5. Thus, about the same collagen forming effects are apparent in Examples 5 through 7 as in Comparative Examples 3 through 5. Also, a comparison of viable cell counts by the testing of cytotonic effects indicates that whereas high values of 1.39 to 1.72 at a concentration of 0.01%, and 1.40 to 1.97 at a concentration of 0.05% are obtained in Examples 5 through 7, the values in Comparative Examples 3 through 5 are low at 1.08 to 1.26 when the concentration is 0.01%, and 1.20 to 1.66 when the concentration is 0.05%. Thus, it is apparent that Examples 5 through 7 have better cytotonic effects than do Comparative Examples 3 through 5.

It can also be seen in Table 3 that the control not treated with the cosmetic material of Example 6, which is the cosmetic material of the present invention, had a bacteriostatic activity of −0.9, and yet the viable bacterial count was increased, and no bacteriostatic effects were identified by the antibacterial action test. In contrast, a sample treated with the cosmetic material of Example 6 had a bacteriostatic activity of 0.8, which was lower than the standard evaluation value (2.2) for bacteriostatic activity, but because the viable bacterial count was prevented from increasing, it was apparent that bacteriostatic effects had been achieved.

Tyrosine is hydroxylized into dopa by tyrosinase, and dopa is furthermore oxidized by tyrosinase and converted to dopaquinone. Dopaquinone is oxidized into dopachrome by auto-oxidation via leukodopachrome, and ultimately becomes melanin. Thus, by inhibiting tyrosinase activity, melanin formation is inhibited, and it is suggested that whitening effects are thus obtained; therefore, the tyrosinase inhibition rate becomes an indicator of the inhibition of melanin formation in vitro. Also, as shown in Table 4, no tyrosinase inhibiting action was identified in the cosmetic material of any of Comparative Examples 3 through 5. In contrast, tyrosinase inhibition rates of 65% and 72% were identified in the cosmetic materials of Examples 5 and 6, showing potent tyrosinase inhibiting action, with apparent usefulness as a cosmetic material having whitening effects.

It is apparent from the above results that the cosmetic material obtained by the manufacturing method of the present invention has excellent SOD activity, cytotonic effects, collagen-forming effects, antibacterial effects, and tyrosinase inhibiting action not as a result of any single component thereof, but of the combined effects of the various components contained therein. Particularly, because the viable cell count, collagen quantity, SOD activity, and tyrosinase inhibiting action were all higher in Example 6, which was obtained by culturing with Bacillus natto, than in Examples 5 and 7, it was apparent that Example 6 had particularly excellent SOD activity, cytotonic effects, collagen-forming effects, antibacterial effects, and tyrosinase inhibiting action.

1. A method for manufacturing a cosmetic material, characterized in adding beans to a water-based solvent and performing extraction, and then microfiltering and/or fraction purifying the bean extract thus obtained.

2. A method for manufacturing a cosmetic material, characterized in adding beans to a water-based solvent and performing extraction, inoculating microorganisms belonging to the genus Bacillus into the bean extract thus obtained, and fermenting and culturing the product.

3. The method for manufacturing a cosmetic material according to claim 2, wherein the microorganisms belonging to the genus Bacillus comprise Bacillus natto or Bacillus subtilis.

4. A method for manufacturing a cosmetic material, characterized in adding beans to a water-based solvent and performing extraction, and then employing enzymes to act on the bean extract thus obtained.

5. The method for manufacturing a cosmetic material according to claim 4, wherein the enzymes comprise proteases derived from soybean Aspergillus.

6. A cosmetic material characterized in being obtained by adding beans to a water-based solvent and performing extraction, and then microfiltering and/or fraction purifying the bean extract thus obtained.

7. A cosmetic material characterized in being obtained by adding beans to a water-based solvent and performing extraction, inoculating microorganisms belonging to the genus Bacillus into the bean extract thus obtained, and fermenting and culturing the product.

8. A cosmetic material characterized in being obtained by adding beans to a water-based solvent and performing extraction, and then employing enzymes to act on the bean extract thus obtained.
